(19) United States
(12) Patent Application Publication
(10) Pub. No.: US 2005/0137499 A1
(43) Pub. Date: Jun. 23, 2005

(54) Ductal lavage catheter

(76) Inventors: Ellen E. Sheets, Concord, MA (US); Arthur L. Smith, Tyngsborough, MA (US)

Correspondence Address: CYTYC CORPORATION, 250 CAMPUS DRIVE, MARLBOROUGH, MA, 01752, US

(21) Appl. No.: 10/746,940

(22) Filed: Dec. 23, 2003

(51) Int. Cl. A61M 21/00; A61M 1/06; A61B 10/00

(52) U.S. Cl. 600/562; 600/27; 604/73

(57) ABSTRACT

A ductal access device is provided for accessing a breast duct and collecting biological material from within the duct. The access device may include an elongated member having an outer diameter sized for positioning within the breast duct and an internal lumen for infusing a fluid into the breast duct and collecting fluid from the breast duct and adapted to slideably receive a ductal introducer. The introducer may extend beyond the distal tip of the catheter and serve to penetrate the ductal orifice.

### FIELD OF THE INVENTION

The present invention relates to a medical instrument having at least a portion that is introduced into the body of a mammal in order to perform diagnostic or therapeutic medical procedures. Specifically, aspects of the present invention relate to a catheter for introducing into a breast duct through a ductal orifice.

### BACKGROUND OF THE INVENTION

The breast is a specialized, glandular structure including a system of complicated breast ducts that radiate from the nipple and that are bound together by fairly dense connective tissue. Each of these breast ducts includes an associated ductal orifice on the surface of a nipple through which ductal fluid may be expressed. Each duct includes a series of successive interlobular branches that drain through the main, lactiferous branch, which terminates and exits the breast at the nipple via the associated ductal orifice. Immediately proximate the ductal orifice, each lactiferous duct includes a lactiferous sinus in which ductal fluid may accumulate. A ductal sphincter resides within the lactiferous sinus and prevents ductal fluid from unintentionally exiting the breast duct through its associated ductal orifice.

Breast cancer is believed to begin in the lining of these breast ducts. For several decades significant members of the medical community dedicated to studying breast cancer have believed and shown that the cytological analysis of cells retrieved from nipple discharge fluid from within breast ducts may provide valuable information leading to identifying patients at risk for breast cancer. Indeed, Papanicolaou contributed to the genesis of such a possibility of a “Pap” smear for breast cancer by analyzing the cells contained in nipple discharge. More recently, cancer specific markers have been detected in ductal fluid obtained by nipple aspiration. However, the retrieval techniques and instruments used by these members of the medical community did not routinely obtain meaningful ductal fluid samples.

In their attempts to retrieve the breast duct fluid sample including ductal epithelial cells, practitioners introduced wash fluids into a breast duct using indwelling hair-like single lumen catheters. After the fluid was introduced into the duct, the fluid introduction catheters were removed. Then, externally applied nipple aspiration techniques or external pressure applied to the breast were used to collect samples of the ductal fluid. However, these techniques required that significant, sometimes painful, pressure be created on the nipple surface or along the sides of the breast to overcome the fluid retaining properties of the ductal sphincter. Also, these techniques did not routinely provide meaningful ductal fluid samples with a sufficient number of ductal epithelial cells for a meaningful cellular analysis. These techniques typically caused the recovery of samples with twenty or fewer ductal epithelial cells. Additionally, these techniques did not provide samples with cell clusters of 10 or more cells. As a result, the obtained fluid samples could not consistently provide an accurate indication of whether or not the duct from which they were retrieved included precancerous or cancerous cells. Consistent, meaningful ductal epithelial cell samples have been provided by the medical instrument disclosed in U.S. Pat. No. 6,413,228 to Hung et al. that is hereby incorporated by reference in its entirety.

The human breasts are composed of fatty tissue, fibrous tissue, breast ducts and milk glands. Human breasts are believed to contain from 6 to 8, or more breast ducts. The ductal lavage procedure discussed above, and sampling results may be greatly effective in screening patients for an early warning of breast cancer risk. However, in performing the ductal lavage procedure, a physician may have difficulty inserting the catheter into a breast duct. The breast duct is a complex anatomical pathway to the breast milk glands. The physician must access the breast duct so as not to cause damage to the inner walls of the duct and/or avoid puncturing the duct. However, it is believed the deeper a catheter is inserted into breast duct, the greater the risk of puncturing the breast duct walls. Therefore, a need exists for a ductal access catheter that allows the physician to adjust its flexibility and rigidity so as to adapt the catheter to the ductal geometry and direct the catheter deep into branches of the ductal network.

During the ductal access procedure, a catheter is inserted into a duct opening in the nipple that may cause some discomfort to the patient. Thus, improved insertions systems and methods for ductal access are needed.

### SUMMARY OF THE INVENTION

Aspects of the present invention pertain to systems and methods for intraductal access and navigation, including a catheter having different distal tip configurations and an introducer having different lengths. In accordance with one aspect of present invention, a device is disclosed for being introduced and positioned within a breast duct for introducing or removing material within the breast duct, the apparatus comprising a manifold hub comprising a plurality of port openings in fluid communication with an interior of the manifold hub, at least two of the openings being in fluid communication with a pair of elongated channels that extend through a portion of the manifold hub and a catheter extending from the manifold hub, the catheter comprising an elongated internal lumen extending substantially parallel to a longitudinal axis of the catheter, said lumen being in fluid communication with the manifold hub and adapted to slidiably receive a ductal introducer. The introducer may extend at least approximately 0.250, 0.492, or 0.984 inches beyond the distal tip of the catheter. The distal tip of the catheter may have an atraumatic configuration.

In yet another aspect of the present invention, a device is disclosed for being introduced and positioned within a breast duct for introducing or removing material within the breast duct, said apparatus comprising a manifold hub comprising a plurality of port openings in fluid communication with an interior of said manifold hub, at least two of said openings being in fluid communication with a pair of elongated channels that extend through a portion of said manifold hub and a catheter extending from the manifold hub and a sheath which extends internally through both the hub and the catheter, comprising an elongated internal lumen extending substantially parallel to a longitudinal axis of said sheath, said lumen being in fluid communication with said manifold hub and adapted to slidiably receive a ductal introducer therein and said sheath extends beyond the distal tip of said catheter. The distal tip of the sheath may extend at least approximately 1.5 mm, 6.0, or 18.5 millimeters beyond the distal tip of the catheter. The distal tip of the catheter may have an atraumatic configuration.

In still another aspect of the present invention, a method for obtaining cellular material from a human breast milk duct using a ductal access comprising a manifold hub comprising a plurality of port openings in fluid communication with an interior of the manifold hub, at least two of the openings being in fluid communication with a pair of elongated channels that extend through a portion of the manifold hub and a catheter extending from the manifold hub, the catheter comprising an elongated internal lumen extending substantially parallel to a longitudinal axis of the catheter, said lumen being in fluid communication with the manifold hub and adapted to slideably receive a ductal introducer; including the steps of inserting the introducer into the internal passageway of a breast duct; and advancing the catheter into the breast duct.

In still another aspect of the present invention, a method for obtaining cellular material from a human breast milk duct using the device comprising a manifold hub comprising a plurality of port openings in fluid communication with an interior of said manifold hub, at least two of said openings being in fluid communication with a pair of elongated channels that extend through a portion of said manifold hub and a catheter extending from the manifold hub and a sheath which extends internally through both the hub and the catheter, comprising an elongated internal lumen extending substantially parallel to a longitudinal axis of said sheath, said lumen being in fluid communication with said manifold hub and adapted to slideably receive a ductal introducer therein and said sheath extends beyond the distal tip of said catheter; including the steps of inserting the introducer into the internal passageway of a breast duct advancing the sheath into the breast duct and advancing the catheter into the breast duct.

The above and other aspects, features and advantages of the present invention will be readily apparent and fully understood from the following detailed description illustrative embodiments in conjunction with the accompanying drawings, which are included by way of example, and not by way of limitation with regard to the claimed invention.

### BRIEF DESCRIPTION OF THE DRAWINGS

FIG. 1 is a schematic representation of a first ductal access device in accordance with one or more aspects of the present invention;

FIG. 2 is a perspective view of a catheter in accordance with one or more aspects of the present invention;

FIG. 3 is a partial schematic side view of a catheter in accordance with one or more aspects of the present invention;

FIG. 4 is a schematic side view of a catheter with a removable tip in accordance with one or more aspects of the present invention;

FIG. 5 is a schematic representation of a second ductal access device in accordance with one or more aspects of the present invention

FIG. 6 is a sectional view of a catheter shown in FIG. 5 taken along an central axis to illustrate an interior construction and components in accordance with one or more aspects of the present invention;

FIG. 7 is a perspective view of a catheter in accordance with one or more aspects of the present invention;

FIG. 8 is a sectional view of a catheter shown in FIG. 7 taken along an central axis to illustrate an interior construction and components in accordance with one or more aspects of the present invention;

FIG. 8A is a sectional view similar to FIG. 8 showing an alterative embodiment of a catheter in accordance with one or more aspects of the present invention;

FIGS. 9A-9C are diagrams illustrating a method of accessing a breast duct which a catheter of FIG. 2;

FIGS. 10A-10C are diagrams illustrating a method of accessing a breast duct which a catheter of FIG. 7;

FIG. 11 is a table illustrating exemplary parametric values and materials for guide wire and introducers that may be implemented with aspects of the present invention;

FIG. 12 is a table illustrating exemplary parametric values and materials for cannula that may be implemented with aspects of the present invention; and

FIG. 13 is a partial schematic side view of a catheter in accordance with one or more aspects of the present invention.

FIG. 14 is a schematic representation of an alternative ductal access device in accordance with one or more aspects of the present invention.

FIG. 15 is a schematic representation of an alternative ductal access device in accordance with one or more aspects of the present invention.

FIG. 16 is a schematic representation of an alternative ductal access device in accordance with one or more aspects of the present invention.

### DETAILED DESCRIPTION OF ILLUSTRATIVE EMBODIMENTS

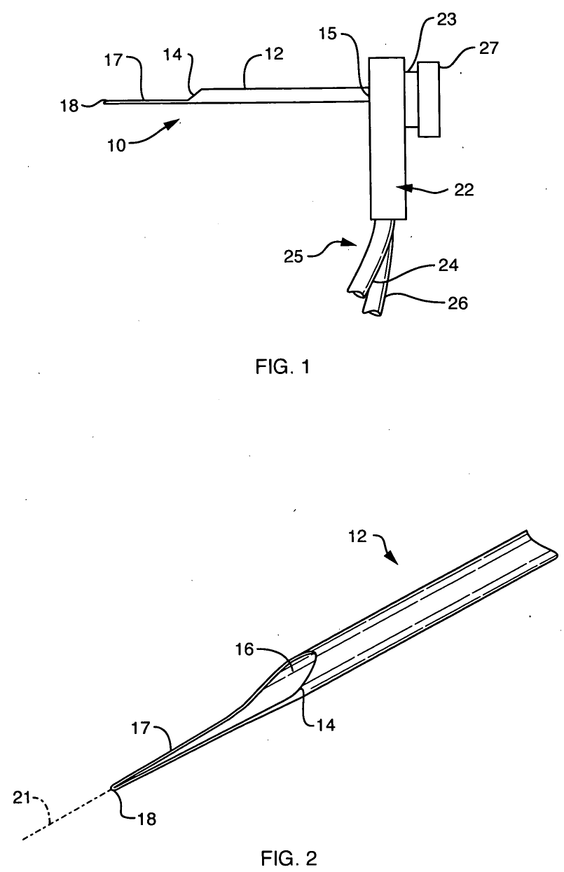

FIGS. 1-3 illustrate preferred embodiments of an inventive access device 10 for accessing a body orifice, such as a breast duct. The access device 10 comprises a catheter 12 having a distal end 14 with a centrally disposed single lumen 16 which extends the length of the catheter 12, and an elongated distal tip portion 17 which extends beyond the distal end 14 a predetermined distance. The center axis 21 of distal tip portion 17 extends substantially parallel to the center axis 19 of single lumen 16 and is disposed within the wall of the catheter. In such an exemplary construction, the distal tip portion 17 is disposed “off-axis” or “off-center” from center axis 19 of lumen 16. The off-axis construction of distal tip portion 17 advantageously maintains the open inner diameter of the catheter for maximum fluid flow in and out of the breast duct. Further, the off axis configuration allows substantially improved flow because it does not to interfere with the introduction or removal of fluid and collected samples so not to compromise the ability to retrieve a meaningful sample while still being in direction of the catheter and lumen. The elongated configuration of the distal tip portion 17 acts as an effective guide while not interfering with fluid infusion or compromising ductal fluid sample collection. Distal tip portion 17 also provides ease of insertion of catheter 12 in the ductal orifice. In one construction, distal tip portion 17 is flexible in nature so as to allow the catheter to traverse the potentially tortuous and/or angled geometry of the human breast duct. Further, distal tip portion 17 distends the ductal orifice to reduce the associated pain upon insertion of the catheter therein.

Catheter 12 has dimensions which permit introduction of the distal end 14 through a ductal orifice and positioning a distal end thereof distal to the ductal sphincter of a human breast. In one construction, the catheter 12 has a maximum outer diameter of approximately 0.039 inches so as to cannulate the ductal orifices of the breast. Nevertheless, other dimensions are possible for the outer diameter (e.g., approximately 0.025 to 0.039 inches). Single lumen 16 with an internal ID of approximately 0.025 inches accesses the breast duct and through which fluid is infused, and from which ductal fluid samples including ductal epithelial cells are collected or drawn up out of the duct. Distal tip portion 17 has dimensions which permit introduction through a ductal orifice so as to lead the catheter 12 into the breast duct. In one exemplary construction, distal tip portion 17 has a smaller diameter than the catheter 12 to allow ease, of insertion into the breast duct. Distal tip portion 17 may have a diameter of approximately 0.010 inches, although other dimensions are possible (e.g., 0.008 to 0.012 inches). In an alternative construction, the distal tip portion 17 may be flexible.

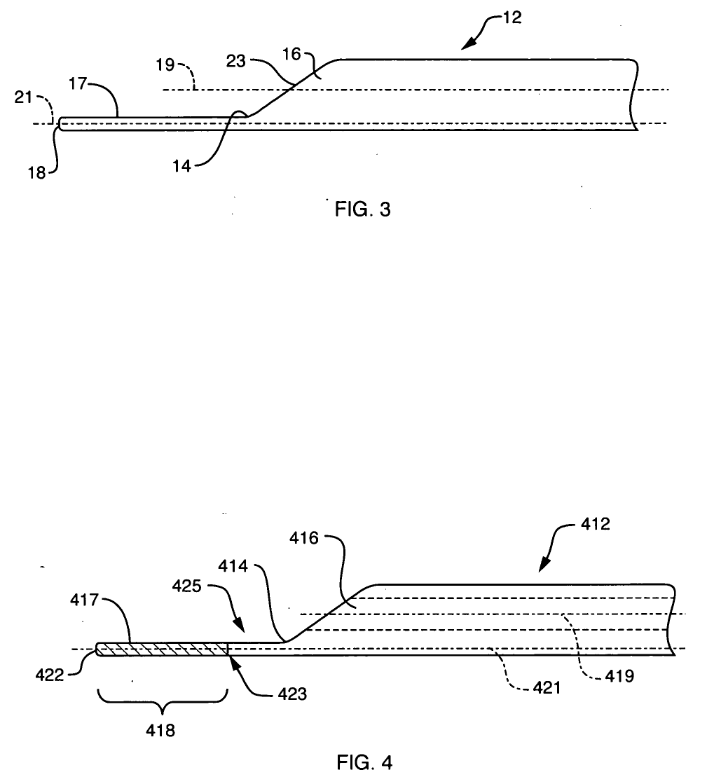

Referring to FIGS. 2 and 3, the distal tip portion 17 may have a tapered configuration being largest at the distal end 14 of the lumen 16 (i.e., proximal end of distal tip 17) extending therefrom to be smallest at the distal end 18. In use, the taper and flexibility of the distal tip portion 17 guides the catheter for intraductal movement. Shown in FIG. 3, the transition between the distal tip portion 17 and catheter 12 includes a beveled surface 23. This beveled surface 23 provides a smooth transition between distal tip portion 17 of catheter 12, which makes the catheter 12 easy to introduce into the ductal opening after the insertion of the tip portion 17 into the breast duct. Hence, the distal tip portion 17 may hold the duct opening in position so that the catheter 12 enters with relative ease. As may be appreciated by one of ordinary skill in the art, the beveled surface 23 reduces the stress level on the tissue being penetrated and spread open by the distal end 14 of the catheter 12. In contrast to traditional catheters, discomfort to the patient is greatly reduced with the access device 10 of the present invention. The edges of the catheter at the distal end and the end 18 of the distal tip 17 may include an atraumatic configuration. In one atraumatic configuration, the edges and end 18 are rounded to reduce friction and eliminate surfaces that could puncture or otherwise injure the duct. Thus, this construction reduces localized trauma to the tissue verses non-atraumatic designs.

Referring to FIG. 1, a multiport hub 22 is coupled to the proximal end 15 of catheter 12. In a preferred construction, hub 22 includes transparent material so that the user may visualize the flow to and from the breast duct during a lavage procedure. In another construction, hub 22 has a low profile so as to reduce the torque on the breast nipple after insertion of catheter 12. This overcomes the excessive generated torque on the breast nipple known to cause obstruction of ductal fluid due to compression of the tissue. Thus, improved collection of ductal cellular material is provided.

Hub 22 is attached to a tubing set 25 which comprises an infusion tube 24 from which fluid is infused into lumen 16 through hub 22 and a collection tube 26 from which fluid is collected from lumen 16 via hub 22. Infusion tube 24 and collection tube 26 are attached to hub 22 in a conventional manner to allow fluid flow. In one construction, infusion tube 24 and collection tube 26 are translucent and have standard luer connections at their distal ends that mate with ports on the hub 22 and receive fluids. The proximal ends of the tubes 24, 26 also include standard luer connection that a practitioner or attendant to manage the various ductal fluids using an appropriate syringe with a standard male luer. If desired, tubes 24, 26 may be labeled to indicate the inflow and outflow paths, e.g. infusion or collection functions.

In a further construction, an optional pinch clamp or other flow control device (not shown) may be disposed on the outflow tube, collection tube 26. In use, the clamp closes the collection tube 24 to prevent fluid leakage from the tubing during fluid infusion into a cannulated breast duct. In one construction, hub 22 includes an ergonomic handle 27 for the user to grasp during a ductal lavage procedure. It should be recognized that a fluid used in the hub 22 and catheter 12 may be virtually any appropriate fluid for the ductal lavage procedure. Exemplary ductal wash fluids which may be used with hub 22 includes but is not limited to saline, phosphate buffered saline, a nonabsorbable fluid, an isotonic solution, an osmotic solution, a hypotonic solution, and a hypertonic solution. Nevertheless, an appropriate therapeutic fluid may be provided by way of the ductal access devices describes herein. Alternatively, the fluids could include diagnostic or therapeutic fluids carrying diagnostic and/or therapeutic agents.

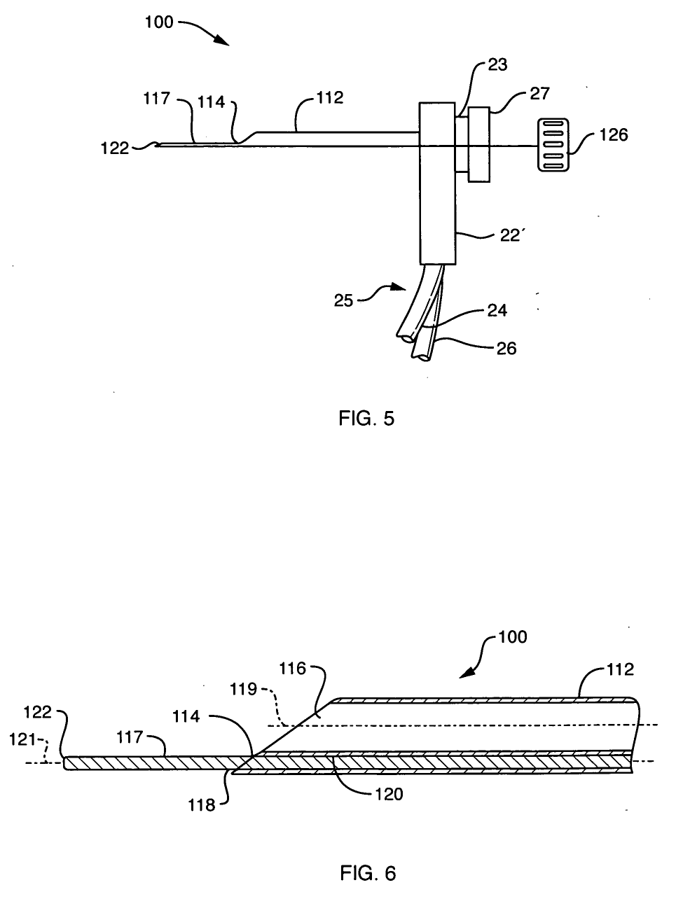

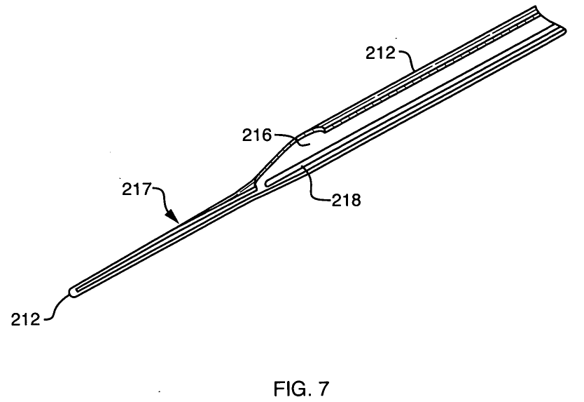

In an alternative construction depicted in FIGS. 5 and 6, an inventive ductal access system 100, includes a catheter 112 having a distal end 114 with a centrally disposed single lumen 116 which extends the length of the catheter 112. Catheter 112 includes a tubular introducer pathway 120 adapted to slidably receive a ductal introducer 118 therein. Long axis 121 of pathway 120 is offset but substantially parallel to long axis 119 of lumen 116. For ease of explanation, as used herein the term “introducer” is defined to include guidewires, dilators, stylettes or portion of any of these that may be inserted within a passageway of a catheter and into a ductal orifice. Ductal introducer 118 is provided for ease of varying the length of tip portion 117 so as to improve intraductal travel of the catheter 112 within the breast duct and to reduce risk of puncturing the ductal wall and rupturing the breast duct. By removably inserting the ductal introducer 118 into the axis 121 of pathway 120, the user is able to choose an introducer having a desired stiffness for the particular patient. Additionally, this enables the practitioner or attendant to change the flexibility and stiffness of the introducer 118. The stiffness and flexibility may be a function of a material property and/or cross-sectional shape. One example of a material property that relates to flexibility is the modulus of elasticity. In another example, the cross-sectional shape of the introducer 118 may be circular, elliptical, oval or other shapes that provide predetermined stiffness in one or more directions. One of skill in the art would recognize these various shapes relate to a section modulus of the introducer 118. Therefore, it should be recognized that introducer pathway 120 may be virtually any appropriate cross-sectional shape to meet the cross-sectional shape of the introducer 118.

Ductal introducer 118 includes a distal end 122, which enters the breast duct. The opposing proximal end 124 of ductal introducer 118 includes a handle 126. Handle 126 provides ease of operation so that a user may grasp and manipulate the introducer 118 to access and navigate the breast duct anatomy. Ductal access device 100 includes a hub 22′ which has similar construction and components as hub 22 shown in FIG. 1. In hub 22′, the introducer pathway 120 extends through so that the introducer 118 may be inserted. The pathway may be a tube extending through the exterior of the hub 22′ or the tube may be located on the outer surface of the hub. An adjustable tip portion 117 of introducer 118 is defined from the distal end 122 of ductal introducer 118 to the distal end portion 114 of catheter 112. In this configuration, the length of the tip portion 117 is selectively adjustable by the user for accessing and traversing the breast duct. In one aspect, ductal introducer 118 may be embodied as a guide wire, which is easily inserted into pathway 120 and into the breast duct. In one construction shown in FIG. 6, the center axis 121 of introducer pathway 120 extends substantially parallel to the center axis 119 of single lumen 116. As discussed above, in such an exemplary construction, the introducer pathway 120 is disposed “off-axis” or “off-center” from center axis 119 of lumen 116. Thus, ductal introducer 118 with the off-axis construction of catheter 112 provides similar benefits as the ductal access device 10.

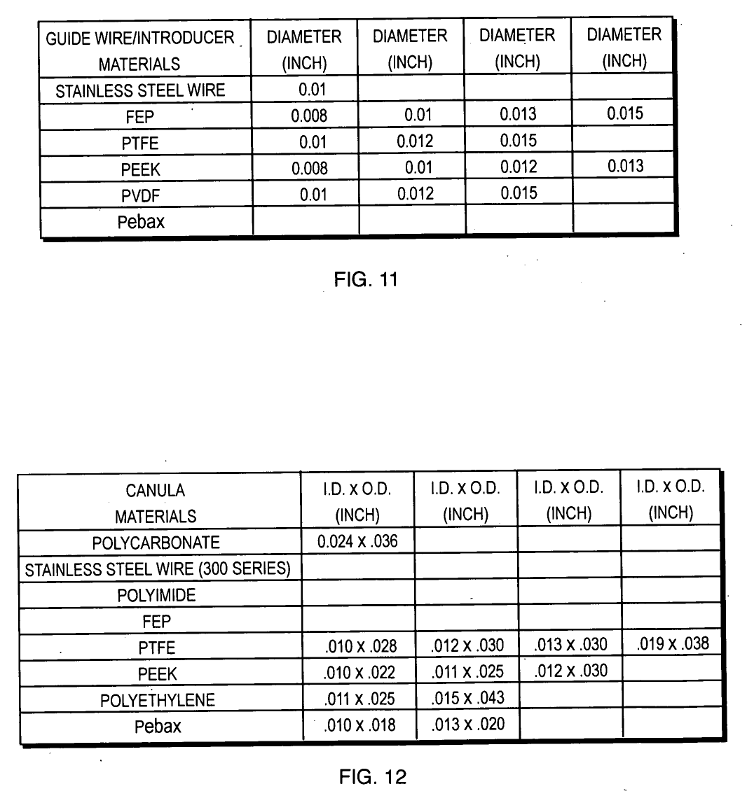

The introducers described herein may be formed of appropriate cross-sectional shapes and various materials for medical use. The shapes and materials enable desired rigidity and flexibility for intraductal movement. Alternative materials for the introducer 118 may include but are not limited to: Stainless Steel Wire; FEP; PTFE; PEEK; and PVDF and PEBAX. Nevertheless, other appropriate materials may be employed. Specific dimensional value introducers are shown in FIG. 11, but are provided by way of example. Alternate coatings for the introducer and/or catheter may include but are not limited to: hydromer coating; STS SLIP-COAT; MDX; silicone dry; silicone lubricant; PTFE coatings. The specific thickness of the coatings and application may be readily determined by one of ordinary skill in the art.

The introducer may be made of metal or plastics and may have a tapered and/or an atraumatic tip for gently probing and accessing a breast duct. In one example, the introducer may be constructed of a superelastic or shape memory material. As used herein, the term “superelastic shape memory material” refers to a class of metal alloys that have a stress-induced phase change or temperate from austenite to martensite and upon stress release, the material springs back to this original phase and shape. The material structure of a superelastic shape memory material regarding austenite and martensite is well known to one of ordinary skill in the metallurgy art. A NiTi material or NiTi alloy may be used as an alloy material for the introducer. As used herein, a NiTi superelastic shape memory material refers to an alloy that is an intermetallic compound of nickel and titanium having nearly equal mixtures as measured by weight. One composition of a NiTi superelastic shape memory material generally has a greater percentage of nickel by weight than titanium, such as 51%-56% of nickel, and preferably 54-55% nickel. The specific percentages of nickel and titanium may be adjusted by one of ordinary skill in the art.

It is not necessary for the introducer to be composed The introducer may also be formed of a stiff material such as a metal wire or a flexible material such as plastic. In an embodiment of the invention, the combined introducer may be formed of Nitinol due to its flexibility, durability, and biocompatibility. In an alternative embodiment, the introducer may be formed of multiple materials or the same materials having different stiffnesses. As a result, the introducer may have sections that are more flexible than adjacent sections of the same introducer. As a result, for example, the introducer may have a first, stiff portion for guiding the introducer within the ductal lumen and a second, flexible portion that allows the introducer to conform to the shape of the ductal lumen or lumen branch into which it is introduced. In any of the above-discussed embodiments, the introducer may be coated with a liquid or dry lubricant material that reduces the friction between the introducer and the breast duct during the introduction and advancement of the introducer in the duct.

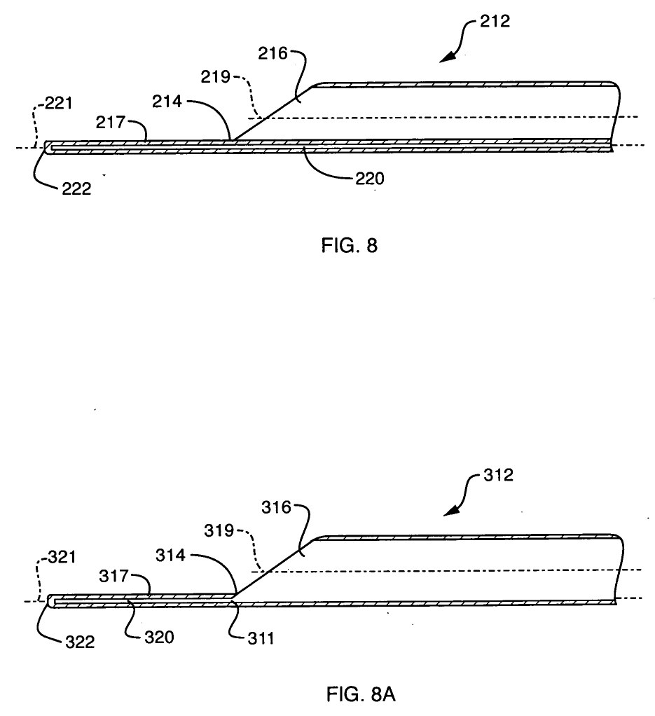

FIGS. 7 and 8 illustrate an alternative construction of an inventive catheter body 212 having a distal end 214 with a centrally (about axis 219) disposed single lumen 216 that traverses the length of the catheter body. In an off-axis configuration, an elongated flexible tip 217 extends beyond the distal end 214 along axis 221. The tip 217 is configured to receive a removable introducer 218. This feature is achieved in that the tip 217 and catheter body 212 include an internal passageway 220 adapted to slidably receive the introducer 218. The passageway 220 may be tubular at the tip 217 and is located in the walls of catheter body 212. Passageway 220 has a closed distal end 122, which is in the initial portion that enters the breast duct. The passageway 220 is opened at the proximal end 211. The closed end feature enables the practitioner or user to adjust the flexibility of the distal 217 during cannulation of the breast duct but prevent fluid from entering the passageway 220. Further, the closed end feature allows the introducer 218 to be removed and another introducer inserted therein when the catheter 212 is positioned within the breast duct. Thus, introducer 218 may be removed or inserted into the tip 217 to selectively adjust the flexibility of tip 217. This allows the user to insert the introducer 218 making the flexible tip more rigid for insertion into the duct, or remove the stylette making the tip more flexible so the tip may transverse tortuous ductal geometry. In this configuration, passageway 220 extends the length of the lumen 216 and is parallel thereto. Advantageously, an aspect of the present invention provides an option to change to tip flexibility which greatly aids the cannulation of the breast duct. The material and thickness of flexible tip 217 may be chosen to achieve a desired stiffness in combination with the flexure characteristics of introducer 218. The closed distal end 222 is rounded to reduce friction between the ductal tissue and tip 217 and prevent the distal end 222 from puncturing duct.

FIG. 8A illustrates an alternative catheter body 312, which has similar construction as catheter body 212 except that a passageway 320 is in the distal tip 317 and not in the walls of the catheter body 312. This allows the user to insert the introducer 218 through the lumen 316 and into the proximal end 311 of the passageway 320. Flexible elongated distal tip 317 includes a closed distal end 322 extending to the distal end 314 of catheter body 312.

In the example of the embodiments shown FIGS. 8 and 8A, the catheter 212, 312 may be provided with a plurality of introducers each of which have a different stiffness characteristic or property. Introducers are provided depending on the portion of duct being accessed. One introducer may be used to introduce the distal tip 217, 317 past the ductal sphincter. Another introducer may be provided to enter a desired branch of breast duct. Yet another introducer, if desired, may be used to enter a final portion of branch. This allows practitioner to design stiffness of tip 217, 317 to match needs presented by different portions of the duct.

An introducer may be designated with a stiffness value to provide certain a stiffness property relative to the other introducers. Purely by way of example, a practitioner may be provided with an introducer having a stiffness value of two, which is designed to be rigid so as to allow penetrating the ductal opening. Another introducer may be provided having a stiffness value four which would be less stiff than a value of two. Yet another introducer may have a value of eight, which would be less stiff than a value of four. In this example, a practitioner is enabled to access the breast duct with a rigid introducer inserted within the passageway 220, 320. The practitioner, while the catheter 212, 312 resides within the breast duct, may remove the rigid introducer and an insert another introducer to continue to advance within the breast duct. Thus, the practitioner is allowed to enter the breast with the catheters 212, 312 without removing the catheter once the breast duct is accessed. Advantageously, catheters 212, 312 allow the practitioner to be more efficient in the ductal access procedure because steps are eliminated or substantially reduced over conventional procedures, e.g., the use of dilators be significantly reduced. Further, patient comfort is increased because less traumatic movement is provided to the breast.

In another construction shown in FIG. 4, a catheter 412 has a distal end 414 with a single lumen 416 disposed about a central axis 419, which extends the length of the catheter, and an elongated distal, tip portion 417, which extends beyond the distal end 414. Catheter 412 has a bevel face and the tip portion 417 includes an extensible introducer 418 formed as a stylette portion. Stylette portion 418 comprises a distal end 422 extending from a distal opening 423 of a transition portion 425 of the lumen 416. Transition portion 425 includes a passageway configured to receive a stylette so that the distal end 422 exits the opening 433. The stylette portion 418 may be made of a flexible metal, plastic material or other materials described above. The stylette portion 418 may be fixed in position and serve as a flexible tip, or it may be removed after insertion of the catheter into the duct. The stylette portion 418 may be removed and replaced with a shorter stylette portion so as to shorten the tip length. Alternatively, the stylette portion 418 may have a longer length. In this manner, the tip length may be adjusted to accommodate the particular duct geometry.

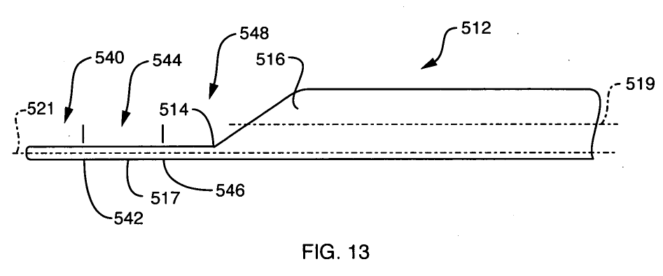

FIG. 13 illustrates an alternative construction of an inventive catheter body 512 having a distal end 514 with a centrally (about axis 519) disposed single lumen 516 that traverses the length of the catheter body. In an off-axis configuration, an elongated tip 517 extends beyond the distal end 514 along axis 521. The elongated tip 517 may have a multi-flexion configuration that has separate regions of different flexions that each correspond to the flexibility, or lack thereof, needed to access a respective portion of a breast duct. This multi-flexion zone configuration provides adaptability for a practitioner to reduce steps for accessing a breast and/or customize the access of the duct to increase patient comfort. In one exemplary example, the elongated tip 517 may have three flex zones to accommodate to access a breast duct. A first flexion zone 540 may extend from the distal end to a first intermediate position 542 along the length of the elongated tip. The first flexion zone 540 may be substantially rigid for entering into the breast duct to overcome the resistance force provided by the tissue at the ductal opening. An adjacent second flexion zone 544 may extend to another intermediate position away from first intermediate position 542 along the length of the elongated tip 517. The second flexion zone 544 may be less rigid than a first flex zone 544 so as to allow the elongated tip 517 to traverse the breast duct geometry. A third flex zone 548 may be provided adjacent to the second flexion zone 544. The third flexion zone 548 may be more flexible than the second flexion zone. In a specific example, the dimensions of the first flexion zone may be 3 cm from the distal end; second flexion zone may have a length of 4 to 8 cm; and the third flexion own may have a length of 2 to 3 cm. Nevertheless, the length of the zones may be configured as desired by the practitioner.

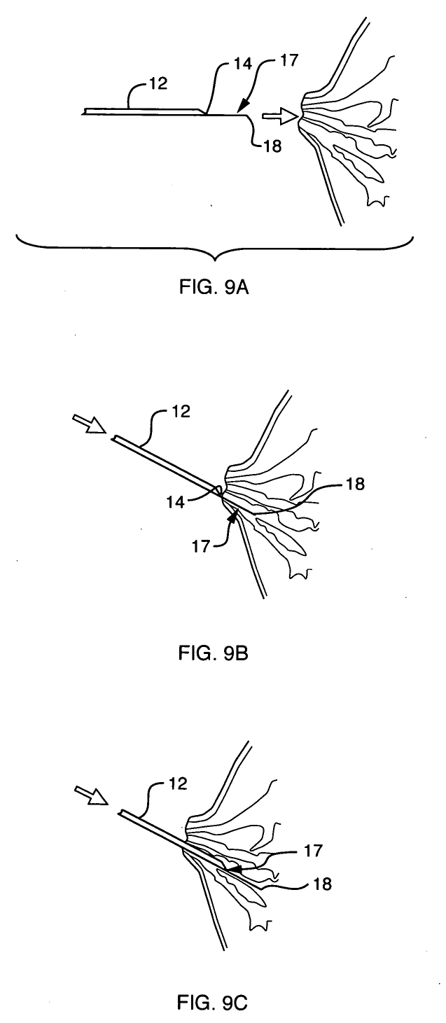

FIGS. 9A-9C are schematic diagrams illustrating a method of accessing a breast duct by way of a catheter 12 of FIG. 2. Referring to FIG. 9A, the catheter 12 is prepared to be introduced into a ductal orifice so that a distal end thereof will be positioned within the duct beyond the ductal sphincter during ductal lavage, introduction of diagnostic materials and/or introduction of therapeutic materials. As shown in FIG. 9B, the distal end 14 of the catheter is outside of the duct. The catheter has a flexible distal tip 17, which provides ease of insertion of catheter 12 in the ductal orifice. Further, the flexible distal tip 17 is inserted into the duct so as to follow the intraductal geometry but not cause damage to the duct. As shown in FIG. 9C, the distal end 14 has entered the duct. The distal tip 17 continues to follow the intraductal geometry to guide the catheter 12 therethrough. Further, the catheter 12 is more rigid than the distal tip 17 so as to straighten the duct for infusion and collection. Although catheter 12 is described with reference to FIGS. 9A-9C, other catheters 112, 212, 312, 412, and 512 may be used in a similar fashion.

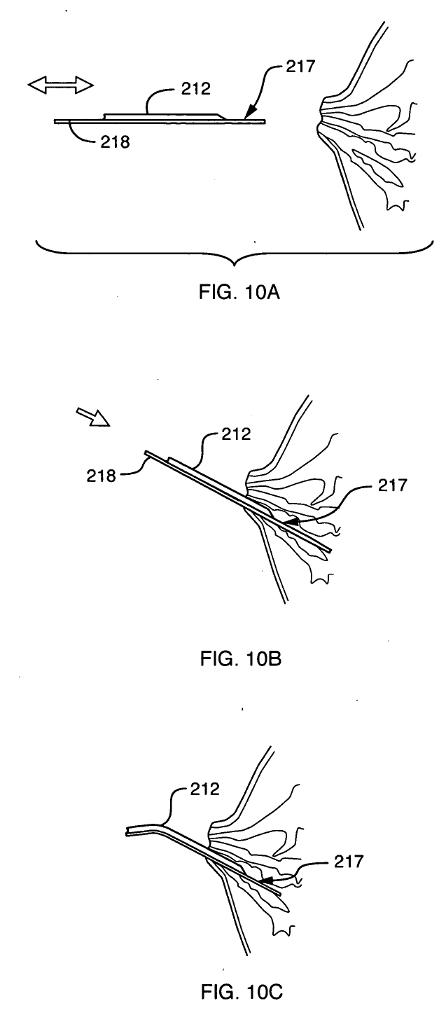

FIGS. 10A-10C are schematic diagrams illustrating a method of accessing a breast duct with a catheter body 212 of FIG. 7. Referring to FIG. 10A, the catheter body 212 is prepared to be introduced into a ductal orifice. The practitioner may choose an introducer 218 of a desired flexibility or rigidity. For example, the introducer 218 of different rigidity properties may be inserted for adjusting to a desired tip characteristic during insertion of the distal tip 217 into the breast duct. This arrangement creates a composite insertion member for accessing the breast duct. The desired tip characteristic may relate to the stiffness and flexibility, which may be functions of a material property or a cross-sectional shape. One of skill in the art would recognize that the composite insertion member may have various shapes and material properties to vary the section modulus.

In particular, the tip 217 may be selectively made rigid to penetrate the duct orifice. In this configuration, the length of the catheter 212 and distal tip 217 is relatively rigid. In FIG. 10B, the introducer 218, which is rigid or somewhat rigid, has been removed from the distal tip 217 while the catheter 212 is inserted in the duct. In this removed configuration, the catheter 212 may flex and bend through the intraductal geometry. If desired, an introducer 218, which is less stiff, may be inserted into the distal tip 217. With or without an introducer 218, the catheter 212 is advanced further into the duct. In FIG. 10C, once a desired position is reached within the duct, a rigid or somewhat rigid introducer 218 may be inserted and advanced into the catheter 212 to straighten the duct for infusion and collection. In a shape memory embodiment, the stiffness or flexibility of introducer 218 may be adjusted in response to the addition of stimulus or removal of a stimulus without removal from the catheter 212. While not shown, it should be recognized that distal tip 217 could be inserted into the ductal orifice without the need of an introducer 218. The introducer of an appropriate stiffness may be inserted into the distal tip 217 during or after intraductal entry of the catheter 212. Although catheter 212 is described with reference to FIGS. 10A-10C, other catheters 112, 312, 412 and 512 may be used in a similar fashion.

As illustrated in FIGS. 5 and 6, the catheter 112 may be tapered along its length to make a smooth transition with a received introducer 118 so that a perceptible transition between the catheter 112 and the introducer 118 that would cause any pain to the patient is not formed and felt by the patient. The catheter 112 may also include an atraumatic distal tip portion 114 at its distal end. The distal tip portion 114 may be tapered, contoured and/or rounded so as to produce an atraumatic tip that will reduce or eliminate trauma to the duct upon entry through the ductal orifice and introduction into the ductal lumen past the ductal sphincter. The distal tip portion 114 may also reduce or eliminate trauma upon withdrawal of the catheter 112 from the duct after the medical procedure, such as ductal lavage or the infusion of a diagnostic and/or treatment agent, has been completed. The tip portion 114 may be composed of a soft polymeric material, e.g. including polyvinyl chloride, polyethers, polyamides, polyethylenes, polyurethanes, copolymers thereof and the like. The tip portion 114 may have a diameter in the range from about 0.012 inches (about 0.031 mm) to about 0.020 inches (about 0.051 mm). In an embodiment, the tip portion 114 has a diameter in the range from about 0.014 inches (about 0.036 mm) to about 0.018 inches (about 0.046 mm). The length of the tip portion 114 (extending from the distal end of the distal portion of the catheter toward the proximal end of the catheter) may be in a range from about 0.10 inch (about 0.25 cm) to about 1.0 inch (about 2.5 cm), more typically in the range from about 0.20 inch (about 0.50 cm) to about 0.70 inch (about 1.8 cm).

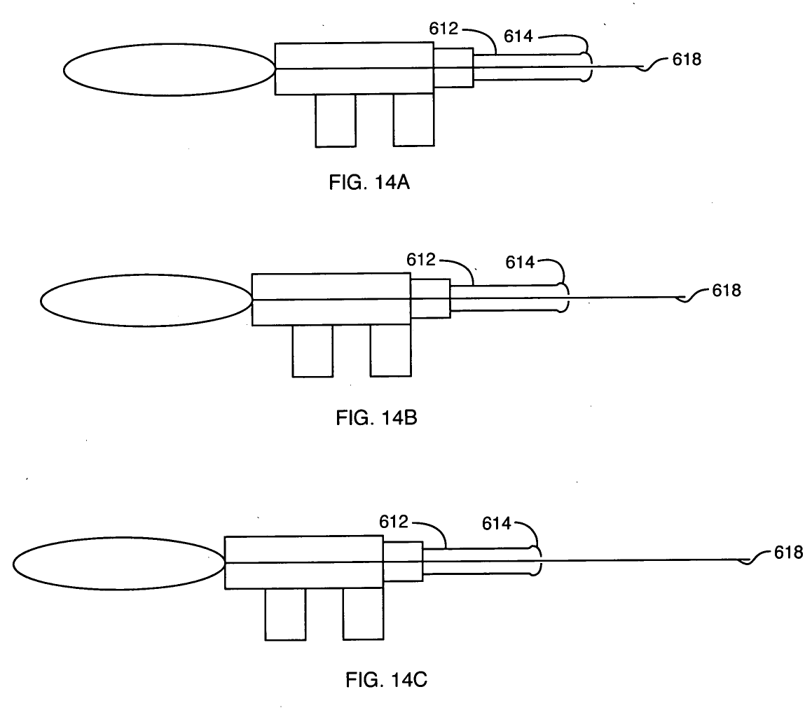

FIGS. 14A, 14B, and 14C depict three different formats of the distal end of an introducer 618. Referring to FIG. 14A, a preferred embodiment of the device is shown in a catheter 612 comprising an elongated internal lumen extending substantially parallel to a longitudinal axis of the catheter and adapted to slidably receive a ductal introducer 618. The distal tip portion 614 of the catheter 612 may be contoured and/or rounded so as to produce an atraumatic tip to form an opening through which the ductal introducer 618 may pass. Such a contour will reduce or eliminate trauma to the duct upon entry through the ductal orifice and introduction into the ductal lumen past the ductal sphincter. The distal tip portion 614 may also reduce or eliminate trauma upon withdrawal of the catheter 612 from the duct after the medical procedure, such as ductal lavage or the infusion of a diagnostic and/or treatment agent, has been completed. The tip portion 614 may be composed of a soft polymeric material, e.g. including polyvinyl chloride, polyethers, polyamides, polyethylenes, polyurethanes, copolymers thereof and the like. The ductal introducer 618 which passes through the internal lumen of the catheter 612, may extend past the distal tip of the catheter 612 and is of sufficient length such that it may penetrate through the ductal orifice and into the ductal lumen past the ductal sphincter prior to the distal tip of the catheter 614 passes through the ductal orifice. The extended length of the probe introducer 618 provides ease of use as well as increasing patient comfort during the ductal lavage procedure. The extended probe introducer 618 may be used to identify the ductal path as well as to introduce the catheter 612 into the ductal orifice. Previously, the ductal lavage process used a separate device (dilator) to identify the ductal path prior to the insertion of the catheter. The extra device and extra procedural step resulted in additional cost, time, as well as an increase in patient anxiety or discomfort due to an additional insertion step to relocate the ductal orifice and ductal path. The increased length of the probe introducer allows the practitioner to locate the ductal path prior to catheter insertion. The catheter may then be inserted into the ductal path by slipping it over the probe introducer without withdrawing the introducer. In an embodiment of the present invention, the length of the ductal introducer that extends beyond the distal tip of the catheter shown in FIG. 14A is at least 0.025 inches.

In FIG. 14B, the introducer 618 extends beyond the distal tip of the catheter 614 at least 0.0492 inches.

In FIG. 14C, the introducer 618 extends beyond the distal tip of the catheter 614 at least 0.0984 inches.

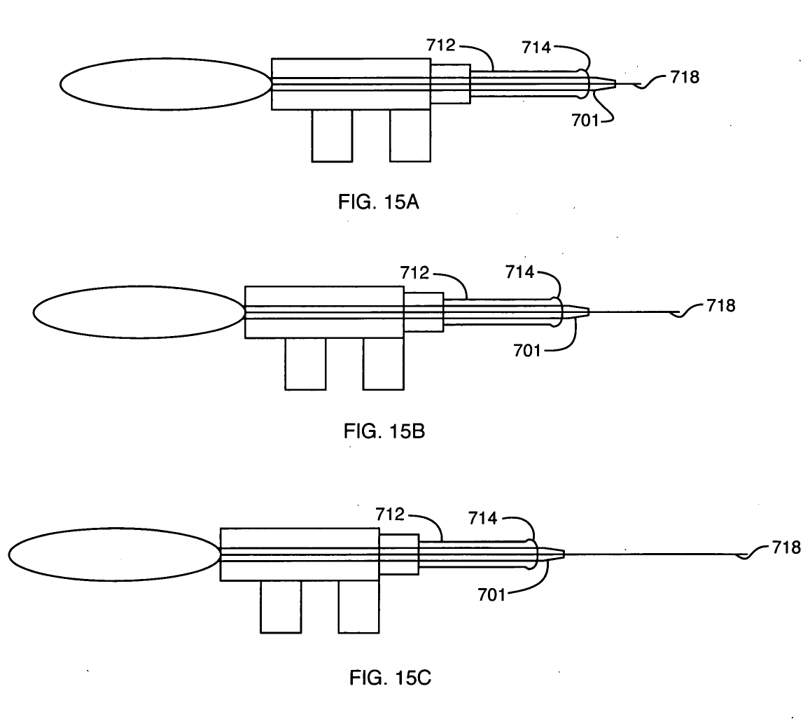

FIGS. 15A, 15B, and 15C depict three different formats of the distal end of a catheter 712. Referring to FIG. 15A, a preferred embodiment of the device is shown in a catheter 712 comprising an elongated internal lumen extending substantially parallel to a longitudinal axis of the catheter and adapted to slidably receive a ductal introducer 718. The distal tip portion 714 of the catheter 712 may be contoured and/or rounded so as to produce an atraumatic tip to form an opening through which the ductal introducer 718 may pass. Such a contour will reduce or eliminate trauma to the duct upon entry through the ductal orifice and introduction into the ductal lumen past the ductal sphincter. The present invention also provides a step transition from the distal tip portion 714 of the catheter to a probe introducer sheath 701. The distal tip portion of the sheath 701 may be contoured and/or rounded so as to produce an atraumatic tip to form an opening through which the ductal introducer 718 may pass. Such a contour will reduce or eliminate trauma to the duct upon entry through the ductal orifice and introduction into the ductal lumen past the ductal sphincter. The distal tip portion may also reduce or eliminate trauma upon withdrawal of the catheter 712 from the duct after the medical procedure, such as ductal lavage or the infusion of a diagnostic and/or treatment agent, has been completed. The tip portion may be composed of a soft polymeric material, e.g. including polyvinyl chloride, polyethers, polyamides, polyethylenes, polyurethanes, copolymers thereof and the like. The ductal introducer 718 that passes through the internal lumen of the catheter 712 and the sheath 701, may extend past the distal tip of the sheath and is of sufficient length such that it may penetrate through the ductal orifice and into the ductal lumen past the ductal sphincter prior to the distal tip of the sheath passes through the ductal orifice. The length of the ductal introducer that extends beyond the distal tip of the sheath shown in FIG. 15A is at least 6.5 millimeters.

In FIG. 15B, the introducer 718 extends beyond the distal tip of the sheath 701 at least 12.5 millimeters.

In FIG. 15C, the introducer 718 extends beyond the distal tip of the sheath 701 at least 25.5 millimeters.

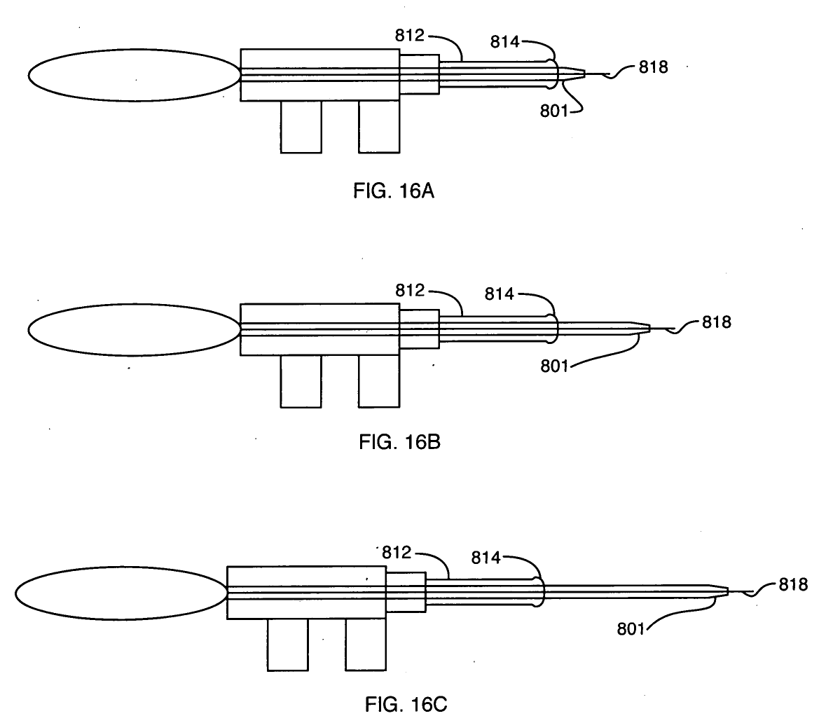

FIGS. 16A, 16B, and 16C depict three different formats of the distal end of a catheter 812. Referring to FIG. 16A, a preferred embodiment of the device is shown in a catheter 812 comprising an elongated internal lumen extending substantially parallel to a longitudinal axis of the catheter and adapted to slidably receive a ductal introducer 818. The distal tip portion 814 of the catheter 812 may be contoured and/or rounded so as to produce an atraumatic tip to form an opening through which the ductal introducer 818 may pass. Such a contour will reduce or eliminate trauma to the duct upon entry through the ductal orifice and introduction into the ductal lumen past the ductal sphincter. The present invention also provides a step transition from the distal tip portion 814 of the catheter to a probe introducer sheath 801. The distal tip portion of the sheath 801 may be contoured and/or rounded so as to produce an atraumatic tip to form an opening through which the ductal introducer 818 may pass. Such a contour will reduce or eliminate trauma to the duct upon entry through the ductal orifice and introduction into the ductal lumen past the ductal sphincter. The distal tip portion may also reduce or eliminate trauma upon withdrawal of the catheter 812 from the duct after the medical procedure, such as ductal lavage or the infusion of a diagnostic and/or treatment agent, has been completed. The tip portion may be composed of a soft polymeric material, e.g. including polyvinyl chloride, polyethers, polyamides, polyethylenes, polyurethanes, copolymers thereof and the like. The probe introducer 801 sheath may extend beyond the distal end of the catheter 812. The extended probe introducer sheath facilitates a smooth transition and entry of the catheter 812 into the ductal orifice. The length of the probe introducer sheath 801 that extends beyond the distal tip of the catheter 812 shown in FIG. 16A is at least 1.5 millimeters.

In FIG. 16B, the probe introducer sheath 801 extends beyond the distal tip of the catheter 812 at least 6.0 millimeters.

In FIG. 16C, the probe introducer sheath 801 extends beyond the distal tip of the catheter 812 at least 18.5 millimeters.

In another embodiment, the probe introducer sheath 801 and the introducer 818, may be combined into a single unit (not shown). The combined sheath/introducer may be formed of a stiff material such as a metal wire or a flexible material such as plastic. In an embodiment of the invention, the combined sheath/introducer may be formed of Nitinol due to its flexibility, durability, and biocompatibility. The combined sheath/introducer may be constructed from a Nitinol wire that is ground to include a tapered small diameter tip. In an alternative embodiment, the combined sheath/introducer may be formed of multiple materials or the same materials having different stiffnesses. As a result, the combined sheath/introducer may have sections that are more flexible than adjacent sections of the same combined sheath/introducer. As a result, for example, the combined sheath/introducer may have a first, stiff portion for guiding the combined sheath/introducer within the ductal lumen and a second, flexible portion that allows the combined sheath/introducer to conform to the shape of the ductal lumen or lumen branch into which it is introduced. In any of the above-discussed embodiments, the combined sheath/introducer may be coated with a liquid or dry lubricant material that reduces the friction between the combined sheath/introducer and the breast duct during the introduction and advancement of the combined sheath/introducer in the duct.

The combined sheath/introducer may be made of metal or plastics, including shape memory metals and plastics, and may have a tapered and/or an atraumatic tip for gently probing and accessing a breast duct. Preferably, a tapered tip will extend distally of the catheter during the introduction of the catheter into the breast duct. After access of the duct is complete, the catheter may be slipped over the combined sheath/introducer and positioned at a location distal to the ductal sphincter.

The catheters as described herein may be constructed of a wide range of appropriate materials for medical use. In a preferred construction, the catheter is formed with PTFE and coated with STS Slipcoat to reduce friction on the device during placement in the breast duct. In lieu of PTFE, alternate materials for the catheter may include but are not limited to: polycarbonate; stainless steel (300 Series); polymide; FEP; PEEK; polyethylene; and PEBAX. Specific dimensional values of catheters are shown in FIG. 12, but are provided by way of example. Nevertheless, other appropriate materials and dimensional values may be employed.

The hub 22 housing may be molded from a polycarbonate. The tubing set 25 may be made of PVC with luer connectors made of polycarbonate. The tubing set may be made of a variety of conventional materials including but not limited to: silicone; low-density polyethylene; PVC; tygon; or polypropylene.

In one manufacturing process, the catheters according to one or more aspects of the present invention may be constructed by using an extrusion process. After the process, the distal end of catheter is shaped by molding and/or cutting. An optional, bevel may be is cut to provide the transition between the distal tip portion and the catheter. The edges of the catheter may be rounded using an RF tipping process known to one of ordinary skill in the art. The catheter may be bonded to the hub using a desired medical grade UV adhesive. In turn, the tubes may be bonded to the hub and to the luer connector using UV adhesive as well. If desired, a pinch clamp may be placed on the outflow tube.

The present invention provides a method for obtaining cellular material from a human breast milk duct includes introducing a ductal access device such as devices 12, 112, 212, 312, 412, and 512 having at least one lumen into a duct. A wash fluid may be introduced through the access device internal lumen into the milk duct. In the method, a volume of at least 2 ml of wash fluid may be present within the duct for a preselected time, and then at least a portion of the wash fluid is collected from the duct through the lumen of the access device. The method preferably further comprises massaging and squeezing the breast tissue after introducing the wash fluid but prior to and/or during collecting a portion of the wash fluid. Introducing the ductal access device typically comprises positioning a distal end of the catheter distal to the ductal sphincter in the breast duct. The access device preferably includes only a single lumen that extends into the duct. The preselected time may be less than one second, but will usually be in the range from one second to one hour.

Accordingly, there are any number of alternative combinations for defining the invention, which incorporate one or more elements from the specification, including the description, claims, and drawings, in various combinations or sub combinations. It will be apparent to those skilled in the relevant technology, in light of the present specification, that alternate combinations of aspects of the invention, either alone or in combination with one or more elements or steps defined herein, may be utilized as modifications or alterations of the invention or as part of the invention. It may be intended that the written description of the invention contained herein covers all such modifications and alterations.

1. A device for being introduced and positioned within a breast duct for introducing or removing material within the breast duct, said apparatus comprising:
a manifold hub comprising a plurality of port openings in fluid communication with an interior of said manifold hub, at least two of said openings being in fluid communication with a pair of elongated channels that extend through a portion of said manifold hub; and
a catheter extending from the manifold hub, said catheter comprising an elongated internal lumen extending substantially parallel to a longitudinal axis of said catheter, said lumen being in fluid communication with said manifold hub and adapted to slidably receive a ductal introducer therein, said introducer extends at least 0.250 inches beyond the distal tip of said catheter.

2. The device of claim 1 wherein said introducer includes, but is not limited to, guidewires, dilators, stylettes or any portion thereof that may be inserted within said internal lumen of said catheter.

3. The device of claim 1, in which said catheter includes a closed distal tip configured to abut a distal end of said introducer.

4. The device of claim 1, in which the introducer extends at least 0.492 inches beyond the distal tip of said catheter.

5. The device of claim 1, in which the introducer extends at least 0.984 inches beyond the distal tip of said catheter.

6. The device of claim 1 in which the distal tip of the catheter has an atraumatic configuration.

7. A device for being introduced and positioned within a breast duct for introducing or removing material within the breast duct, said apparatus comprising:
a manifold hub comprising a plurality of port openings in fluid communication with an interior of said manifold hub, at least two of said openings being in fluid communication with a pair of elongated channels that extend through a portion of said manifold hub; and
a catheter extending from the manifold hub; and
a sheath which extends internally through both the hub and the catheter, comprising an elongated internal lumen extending substantially parallel to a longitudinal axis of said sheath, said lumen being in fluid communication with said manifold hub and adapted to slidably receive a ductal introducer therein and said sheath extends beyond the distal tip of said catheter.

8. The device of claim 7, in which said sheath includes a closed distal tip configured to abut a distal end of said introducer.

9. The device of claim 7, in which the introducer extends at least 0.250 inches beyond the distal tip of said catheter.

10. The device of claim 7, in which the introducer extends at least 0.492 inches beyond the distal tip of said catheter.

11. The device of claim 7, in which the introducer extends at least 0.984 inches beyond the distal tip of said catheter.

12. The device of claim 7 in which the distal tip of the catheter and the sheath has an atraumatic configuration.

13. The device of claim 7 in which the distal tip of the sheath extends at least 1.5 mm inches beyond the distal tip of said catheter.

14. The device of claim 7 in which the distal tip of the sheath extends at least 6.0 mm inches beyond the distal tip of said catheter.

15. The device of claim 7 in which the distal tip of the sheath extends at least 18.5 mm inches beyond the distal tip of said catheter.

16. A method for obtaining cellular material from a human breast milk duct using the device of claim 1 including the steps of:
inserting the introducer into the internal passageway of a breast duct; and
advancing the catheter into the breast duct.

17. A method for obtaining cellular material from a human breast milk duct using the device of claim 7 including the steps of:
inserting the introducer into the internal passageway of a breast duct;
advancing the sheath into the breast duct; and
advancing the catheter into the breast duct.
